(19) United States
(12) Patent Application Publication
(10) Pub. No.: US 2010/0159005 A1
(43) Pub. Date: Jun. 24, 2010

(54) COMBINATION OF TRIAZINE DERIVATIVES AND PPARalpha AGONISTS

(76) Inventors: Gérard Moinet, Orsay (FR); Daniel Cravo, Sartrouville (FR); Didier Mesangeau, Combs La Ville (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/160,507

(22) PCT Filed: Dec. 18, 2006
(86) PCT No.: PCT/EP2006/012186

§ 371 Date: Jul. 10, 2008

(30) Foreign Application Priority Data
Jan. 13, 2006 (FR) 06/00345

(51) Int. Cl. A61K 31/53; A61K 9/28; A61P 3/10; A61P 3/04; A61P 3/06; A61P 9/12; A61P 9/10; A61P 27/02; A61P 25/00

(52) U.S. Cl. 424/474; 514/245

(57) ABSTRACT

The present patent application relates to combinations of triazine derivatives and of PPARα agonists.

### FIELD OF THE INVENTION

The present invention relates to a pharmaceutical composition of triazine derivatives or described pharmaceutically acceptable salts thereof with a PPARα agonist, for the manufacture of a medicament that can be used in the treatment of non-insulin-dependent diabetes and pathologies associated with insulin resistance syndrome.

### TECHNICAL BACKGROUND

“Diabetes mellitus” (or diabetes) is one of the most prevalent diseases in the world today. Individuals suffering from diabetes have been divided into two classes, namely type I or insulin-dependent diabetes mellitus and type II or non-insulin-dependent diabetes mellitus (NIDDM). Non-insulin-dependent diabetes mellitus (NIDDM) accounts for approximately 90% of all diabetics, and is estimated to affect 12 to 14 million adults in the United States alone (6.6% of the population). NIDDM is characterised both by fasting hyperglycaemia and exaggerated postprandial increases in plasmatic glucose levels. NIDDM is associated with a variety of long-term complications, including microvascular diseases, such as retinopathy, nephropathy and neuropathy, and macrovascular diseases, such as coronary heart disease. Numerous studies in animal models show a causal relationship between long-term complications and hyperglycaemia. Recent results obtained by the Diabetes Control and Complications Trial (DCCT) and the Stockholm Prospective Study have for the first time demonstrated this relationship in man by showing that insulin-dependent diabetics have a substantially lower risk of development and progression of these complications if they are subjected to tighter glycaemic control. Tighter control is also expected to benefit NIDDM patients.

Hyperglycaemia in the case of NIDDM is associated with two biochemical anomalies, namely insulin resistance and insufficiency of insulin secretion.

The initial treatment of NIDDM is based on a controlled diet and controlled physical exercise, since a considerable number of diabetics are over-weight or obese (˜67%) and since loss of weight can improve insulin secretion and sensitivity to insulin and lead to normal glycaemia.

Patients suffering from a hyperglycaemia that cannot be controlled solely by diet and/or physical exercise are then treated with oral antidiabetics.

A number of categories of oral antidiabetics are currently used in monotherapy for the treatment of NIDDM:
insulin secretion stimulators. They are represented, firstly, by sulfonylureas (SU) and by “glinides”. As regards SUs, mention will be made in particular of carbutamide (Glucidoral®), glibenclamide/glyburide (Daonil®, Euglucan®), glibomuride (Glutril®), gliclazide (Diamicron®), glimepiride (Amarel®) and glipizide (Glibenese®). As regards the “glinides”, mention will be made in particular of repaglinide (NovoNorm®);
agents that reduce glucogenesis, represented by the biguanides. Mention will be made in particular of metformin (Glucophage®, Stagid®);
insulin sensitisers, represented mainly by thiazolidinediones (TZD). Mention will be made in particular of pioglitazone (Actos®) and rosiglitazone (Avandia®);
alpha-glucosidase inhibitors. Mention will be made in particular of acarbose (Glucor®) and miglitol (Diastabol®).

Triazine derivatives with an antidiabetic effect comparable to that of metformin have been described in WO 01/55122.

Moreover, diabetic patients are also known as being an at-risk population as regards the development of cardiovascular pathologies, in particular arteriosclerosis and atherosclerosis. This is partly due to greater susceptibility to factors, such as hyperlipidaemia or hypercholesterolaemia. In May 2002, the recommendations published by the National Cholesterol Education Program (NCEP) state that although reducing the level of low-density lipoprotein cholesterol (LDL cholesterol) in the serum remains the main therapeutic approach, it is also important to identify patients with a low level of high-density lipoprotein cholesterol (HDL cholesterol) and/or high levels of triglycerides. It has in particular been shown that triglyceride-rich lipoproteins originating either from the liver (VLDL) or from the intestine (chylomicrons) present a high atherogenic risk (D. B. Zilversmit, Clin. Chem., 41(1), 153-158, (1995)). The mechanism via which these “bad” lipoproteins develop explains why patients with a high level of triglycerides and a low level of HDL require particular attention. These mechanisms suggest the importance of having available, in the case of diabetic patients, suitable therapeutic approaches and novel medicaments capable of correcting both glycaemic deregulation and lipid imbalance.

The guidelines and recommendations put forward for the treatment of metabolic syndrome suggest focusing on the causes, such as excess weight and obesity by developing physical exercise and weight-control diets.

The level of LDL cholesterol can be reduced using agents, such as 3-hydroxy-3-methylglutaryl-coenzyme A (HMG-CoA) reductase inhibitors. Aspirin for the thrombotic risk and hypertensive agents are also therapeutic approaches used.

As regards the treatment of a high level of triglycerides, the agents most commonly used are PPARα agonists and in particular fibrates. The compounds most commonly used are:
fenofibrate (Lipanthyl®)
bezafibrate (Befizal®)
ciprofibrate (Lipanor®)
gemfibrozil (Lipur®).

Other PPARα agonists are described in WO 97/27847, WO 97/27857, WO 97/28115, WO 97/28137, WO 97/28149 and U.S. Pat. No. 6,008,239.

PPARα represents a subgroup of the family of nuclear receptors known as PPARs (Peroxisome Proliferator Activated Receptors). PPARα is more particularly expressed in tissues capable of catabolising large amounts of fatty acids, such as the liver, the heart and brown adipose tissue. The activated PPARαs form dimers with RXRs (retinoid X receptors) and this heterodimer, on binding to response elements, regulates a certain number of genes involved in intracellular and extracellular lipid metabolism, such as acyl-CoA oxidase, acyl-CoA synthetase and the apolipoproteins A-I, A-II and C-III.

Fibrates have been mentioned above as PPARα agonists. It is known that fibrates reduce the plasmatic level of triglycerides and cholesterol and that, consequently, they are useful in preventing cardiovascular pathologies in the case of dyslipidaemic patients. Furthermore, fibrates, such as gemfibrozil, fenofibrate, bezafibrate and ciprofibrate increase the level of HDL cholesterol.

It has been envisaged that a treatment combining a reduction of glycaemia in parallel with a reduction of the lipidic factors, and in particular of triglycerides, could lead to better control of the risk factors in the case of patients suffering from non-insulin-dependent diabetes and related pathologies, such as macrovascular and microvascular complications, obesity and insulin resistance.

Thus, a combination of metformin with a fibrate that is useful for the treatment of non-insulin-dependent diabetes has been described in EP 1 054 665, the fibrate being chosen from fenofibrate and bezafibrate. However, given the undesirable effects of metformin, it appeared to be important to have available a novel combination that does not have these drawbacks.

The applicant has demonstrated that this problem can be solved with a novel pharmaceutical composition for reducing the glycaemic and lipidic parameters of patients suffering from non-insulin-dependent diabetes and comprising an antidiabetic agent of triazine type, such as those described in WO 01/55122 and a PPARα agonist. Such a pharmaceutical composition has not been described to date. Moreover, entirely unexpectedly, the combinations according to the invention significantly reduce the side effects, such as the gastrointestinal disorders, such as nausea and diarrhoea.

### DESCRIPTION OF THE INVENTION

The present invention thus relates to a novel pharmaceutical composition comprising an antidiabetic agent of triazine type as described in WO 01/55122 and a PPARα agonist with one or more pharmaceutically acceptable excipients.

Preferably, the triazine derivative is represented by the general formula (I):

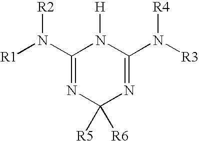

in which:
R1, R2, R3 and R4 are independently chosen from the following groups:
H,
(C1-C20)alkyl optionally substituted by halogen, (C1-C5)alkyl, (C1-C5)-alkoxy or (C3-C8)cycloalkyl,
(C2-C20)alkenyl optionally substituted by halogen, (C1-C5)alkyl or (C1-C5)alkoxy
(C2-C20)alkynyl optionally substituted by halogen, (C1-C5)alkyl or (C1-C5)alkoxy
(C3-C8)cycloalkyl optionally substituted by (C1-C5)alkyl or (C1-C5)-alkoxy
hetero(C3-C8)cycloalkyl bearing one or more heteroatoms chosen from N, O and S and optionally substituted by (C1-C5)alkyl or (C1-C5)alkoxy
(C6-C14)aryl(C1-C20)alkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C6-C14)aryl optionally substituted by amino, hydroxyl, thio, halogen, (C1-C5)alkyl, (C1-C5)alkoxy, (C1-C5)alkylthio, (C1-C5)alkylamino, (C6-C14)-aryloxy, (C6-C14)aryl(C1-C5)alkoxy, cyano, trifluoromethyl, carboxyl, carboxymethyl or carboxyethyl,
(C1-C13)heteroaryl bearing one or more heteroatoms chosen from N, O and S and optionally substituted by amino, hydroxyl, thio, halogen, (C1-C5)-alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,

R1 and R2, on the one hand, and R3 and R4, on the other hand, possibly forming with the nitrogen atom an n-membered ring (n between 3 and 8) optionally containing one or more heteroatoms chosen from N, O and S and possibly being substituted by one or more of the following groups: amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,

R5 and R6 are independently chosen from the following groups:
H
(C1-C20)alkyl optionally substituted by amino, hydroxyl, thio, halogen, (C1-C5)alkyl, (C1-C5)alkoxy, (C1-C5)alkylthio, (C1-C5)alkylamino, (C6-C14)-aryloxy, (C6-C14)aryl(C1-C5)alkoxy, cyano, trifluoromethyl, carboxyl, carboxymethyl or carboxyethyl,
(C2-C20)alkenyl optionally substituted by amino, hydroxyl, thio, halogen, (C1-C5)alkyl, (C1-C5)alkoxy, (C1-C5)alkylthio, (C1-C5)alkylamino, (C6-C14)-aryloxy, (C6-C14)aryl(C1-C5)alkoxy, cyano, trifluoromethyl, carboxyl, carboxymethyl or carboxyethyl,
(C2-C20)alkynyl optionally substituted by amino, hydroxyl, thio, halogen, (C1-C5)alkyl, (C1-C5)alkoxy, (C1-C5)alkylthio, (C1-C5)alkylamino, (C6-C14)-aryloxy, (C6-C14)aryl(C1-C5)alkoxy, cyano, trifluoromethyl, carboxyl, carboxymethyl or carboxyethyl,
(C3-C8)cycloalkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
hetero(C3-C8)cycloalkyl bearing one or more heteroatoms chosen from N, O and S and optionally substituted by amino, hydroxyl, thio, halogen, (C1-C5)alkyl, (C1-C5)alkoxy, (C1-C5)alkylthio, (C1-C5)alkylamino, (C6-C14)-aryloxy, (C6-C14)aryl(C1-C5)alkoxy, cyano, trifluoromethyl, carboxyl, carboxymethyl or carboxyethyl,
(C6-C14)aryl optionally substituted by amino, hydroxyl, thio, halogen, (C1-C5)alkyl, (C1-C5)alkoxy, (C1-C5)alkylthio, (C1-C5)alkylamino, (C6-C14)-aryloxy, (C6-C14)aryl(C1-C5)alkoxy, cyano, trifluoromethyl, carboxyl, carboxymethyl or carboxyethyl,
(C1-C13)heteroaryl bearing one or more heteroatoms chosen from N, O and S and optionally substituted by amino, hydroxyl, thio, halogen, (C1-C5)-alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C6-C14)aryl(C1-C5)alkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
R5 and R6 possibly forming with the carbon atom to which they are attached an m-membered ring (m between 3 and 8) optionally containing one or more heteroatoms chosen from N, O and S and possibly being substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
   or possibly forming with the carbon atom a C10-C30 polycyclic residue optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)-alkoxy, cyano, trifluoromethyl, carboxyl, carboxymethyl or carboxyethyl,
   R5 and R6 together also possibly representing the group ═O or ═S, the nitrogen atom of a heterocycloalkyl or heteroaryl group possibly being substituted by a (C1-C5)alkyl, (C3-C8)cycloalkyl, (C6-C14)aryl, (C6-C14)aryl(C1-C5)alkyl or (C1-C6)acyl group,

and also the racemic forms, tautomers, enantiomers, diastereoisomers and epimers, or mixtures thereof, and the pharmaceutically acceptable salts.

The term “m-membered ring formed by R5 and R6” in particular means a saturated ring, such as a cyclohexyl, piperidyl or tetrahydropyranyl group.

The term “polycyclic group formed by R5 and R6” means an optionally substituted carbon-based polycyclic group and in particular a steroid residue.

One particular group of the invention concerns the pharmaceutical compositions according to the invention in which the triazine derivatives are compounds of the formula (I) in which R5 is hydrogen.

Another particular group of the invention concerns the pharmaceutical compositions according to the invention in which the triazine derivatives are compounds of the formula (I) in which R5 and R6 form with the carbon atom to which they are attached an m-membered ring (m between 3 and 8) optionally containing one or more heteroatoms chosen from N, O and S and possibly being substituted by one or more of the following groups: (C1-C5)alkyl, amino, hydroxyl, (C1-C5)alkylamino, alkoxy(C1-C5), (C1-C5)alkylthio, (C6-C14)aryl, (C6-C14)aryl(C1-C5)alkoxy,

or form with the carbon atom a C10-C30 polycyclic residue optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)-alkoxy, cyano, trifluoromethyl, carboxyl, carboxymethyl or carboxyethyl.

Another particular group of the invention concerns the pharmaceutical compositions according to the invention in which the triazine derivatives are compounds of the formula (I) in which R5 and R6 are independently chosen from the following groups:
(C1-C20)alkyl groups optionally substituted by amino, hydroxyl, thio, to halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl.

A more particular group of the invention concerns the pharmaceutical compositions according to the invention in which the triazine derivatives are compounds of the formula (I) in which R1 and R2 are a methyl group and R3 and R4 represent a hydrogen.

Compounds of the formula (I) that may especially be mentioned include:

| | Formula | Salt |
|---|---|---|
| 1 | 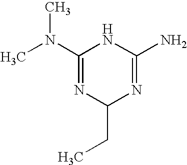 | HCl |
| 2 | 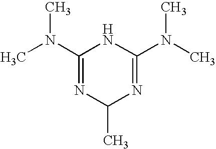 | HCl |
| 3 | 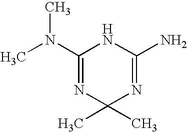 | |
| 4 | 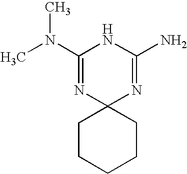 | HCl |
| 5 | 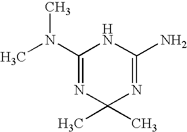 | Methane- sulfonate |
| 6 | 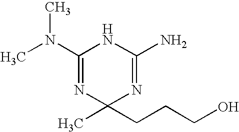 | |
| 7 | 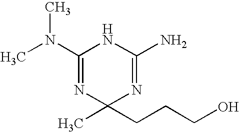 | HCl |
| 8 | 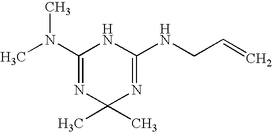 | HCl |
| 9 | 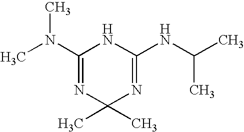 | HCl |
| 10 | 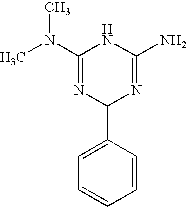 | HCl |
| 11 | 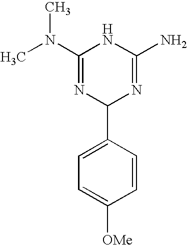 | HCl |
| 12 | 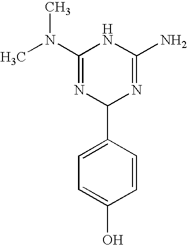 | HCl |
| 13 | 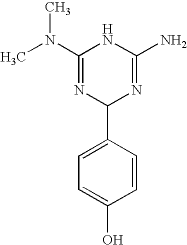 | |
| 14 | 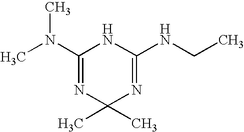 | Fumarate |
| 15 | 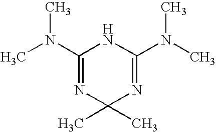 | HCl |
| 16 | 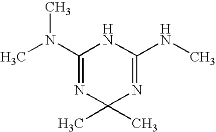 | HCl |
| 17 | 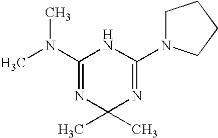 | HCl |
| 18 | 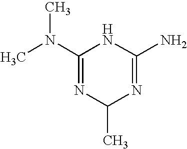 | HCl |
| 19 | 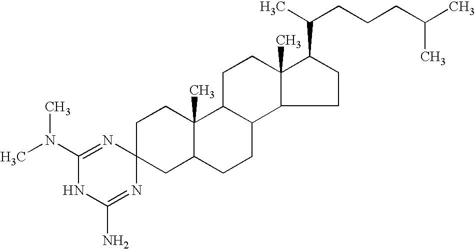 | HCl |
| 20 | 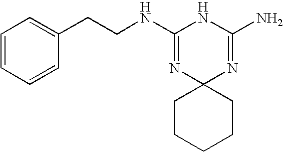 | Carbonate |
| 21 | 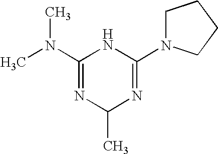 | Carbonate |
| 22 | 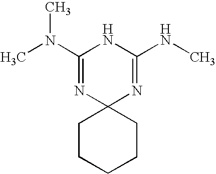 | HCl |
| 23 | 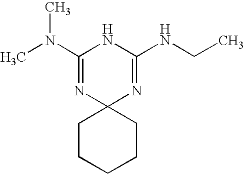 | HCl |
| 24 | 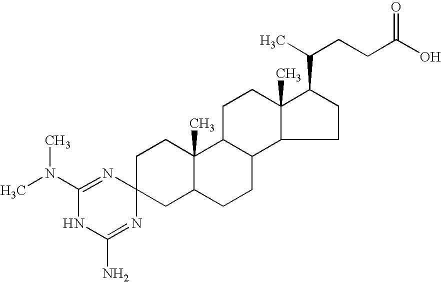 | HCl |
| 25 | 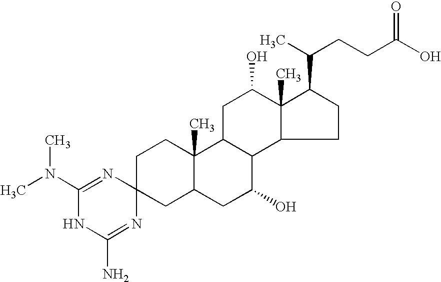 | HCl |
| 26 | 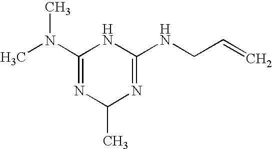 | HCl |
| 27 | 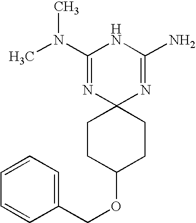 | HCl |
| 28 | 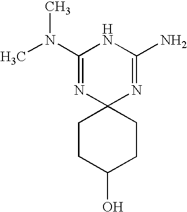 | HCl |
| 29 | 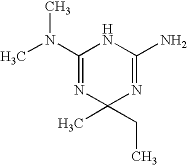 | Carbonate |
| 30 | 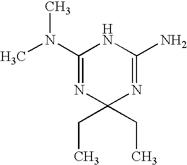 | Carbonate |
| 31 | 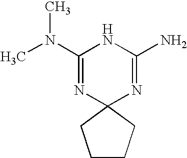 | HCl |
| 32 | 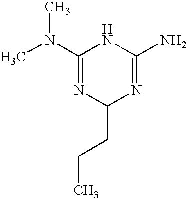 | Carbonate |
| 33 | 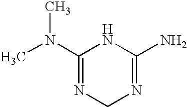 | HCl |
| 34 | 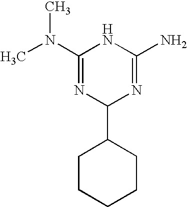 | para-Toluene- sulfonate |
| 35 | 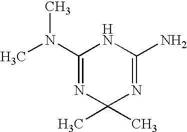 | HCl |
| 36 | 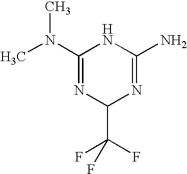 | para-Toluene- sulfonate |
| 37 | 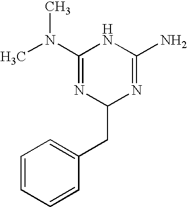 | para-Toluene- sulfonate |
| 38 | 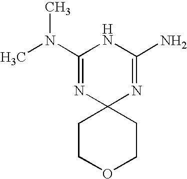 | HCl |
| 39 | 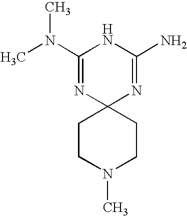 | HCl |
| 40 | 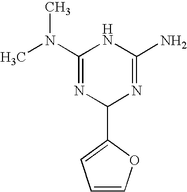 | HCl |
| 41 | 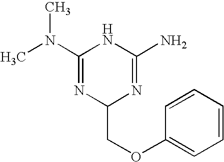 | para-Toluene- sulfonate |
| 42 | 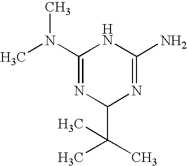 | HCl |
| 43 | 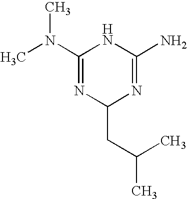 | HCl |
| 44 | 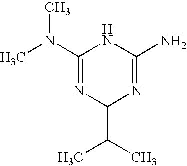 | HCl |
| 45 | 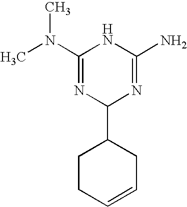 | para-Toluene- sulfonate |

and more preferably the compound of Example 18.

According to yet another preferred embodiment, the invention more particularly relates to pharmaceutical compositions chosen from:
(+)-2-amino-3,6-dihydro-4-dimethylamino-6-methyl-1,3,5-triazine or a corresponding salt thereof with pharmaceutically acceptable organic or mineral acids and fenofibrate;
(+)-2-amino-3,6-dihydro-4-dimethylamino-6-methyl-1,3,5-triazine hydrochloride and bezafibrate;
(+)-2-amino-3,6-dihydro-4-dimethylamino-6-methyl-1,3,5-triazine hydrochloride or a corresponding salt thereof and gemfibrozil;
(+)-2-amino-3,6-dihydro-4-dimethylamino-6-methyl-1,3,5-triazine hydrochloride and ciprofibrate.

Preferably, the PPARα agonist is chosen from all the PPARα agonists generally used in human or veterinary therapy. More particularly, it is chosen from bezafibrate, fenofibrate, gemfibrozil, ciprofibrate and the compounds described in WO 97/27847, WO 97/27857, WO 97/28115, WO 97/28137, WO 97/28149 and U.S. Pat. No. 6,008,239. The PPARα agonists may also be in the form of pharmaceutically acceptable salts, such as, in a non-limiting manner, the hydrochloride, hydrobromide, hydroiodide, sulfate, nitrate, phosphate, citrate, methanesulfonate, trifluoroacetate or acetate, the sodium ion, the potassium ion, the calcium ion or the magnesium ion.

The invention also relates to the tautomeric forms, enantiomers, diastereoisomers and epimers, and mixtures thereof, of the compounds of the general formula (I).

The compounds of the invention of the formula (I) as defined above, containing a sufficiently basic function, or both, may include the corresponding pharmaceutically acceptable salts of organic or mineral acids.

For the purposes of the present invention, the term “corresponding pharmaceutically acceptable salts of organic or mineral acids” means any salt prepared from any non-toxic pharmaceutically acceptable organic or inorganic acid. Such acids include acetic acid, benzenesulfonic acid, benzoic acid, citric acid, carbonic acid, ethanesulfonic acid, fumaric acid, gluconic acid, glutamic acid, hydrobromic acid, hydrochloric acid, lactic acid, mandelic acid, malic acid, maleic acid, methanesulfonic acid, mucic acid, nitric acid, pamoic acid, pantothenic acid, phosphoric acid, succinic acid, tartaric acid and para-toluenesulfonic acid. Hydrochloric acid is advantageously used.

The invention also relates to the chiral salts of the compounds of the formula (I) used for the separation of the racemates of the compounds of the formula (I).

By way of example, the following chiral acids are used: (+)-D-di-O-benzoyltartaric acid, (−)-L-di-O-benzoyltartaric acid, (−)-L-di-O,O′-p-toluoyl-L-tartaric acid, (+)-D-di-O,O′-p-toluoyl-L-tartaric acid, (R)-(+)-malic acid, (S)-(−)-malic acid, (+)-camphanic acid, (−)-camphanic acid, R-(−)-1,1′-binaphthalen-2,2′-diylhydrogenophosphonic acid, (+)-camphoric acid, (−)-camphoric acid, (S)-(+)-2-phenylpropionic acid, (R)-(+)-2-phenylpropionic acid, D-(−)-mandelic acid, L-(+)-mandelic acid, D-tartaric acid, L-tartaric acid, or a mixture of two or more thereof.

The compounds of the formula (I) above also include the prodrugs of these compounds.

The term “prodrugs” means compounds which, when administered to the patient, are chemically and/or biologically converted in the live body into compounds of the formula (I).

In the present description, the terms used have, unless otherwise indicated, the following meanings:
the term “(C1-C20)alkyl” denotes a linear or branched alkyl radical containing from 1 to 20 carbon atoms. Among the C1-C20 alkyl radicals that may especially be mentioned, in a non-limiting manner, are methyl, ethyl, propyl, isopropyl, butyl, sec-butyl, tert-butyl, pentyl, hexyl, octyl, decyl, dodecyl, hexadecyl and octadecyl radicals;
the term “(C1-C20)alkenyl” denotes a linear or branched hydrocarbon-based radical containing one or more unsaturations in double bond form. As alkylene radicals containing from 1 to 20 carbon atoms, mention may be made, in a non-limiting manner, of ethenyl, prop-2-enyl, but-2-enyl, but-3-enyl, pent-2-enyl, pent-3-enyl and pent-4-enyl radicals;
the term “(C1-C20)alkynyl” denotes a linear or branched hydrocarbon-based radical containing one or more unsaturations in triple bond form. As alkylene radicals containing from 1 to 20 carbon atoms, mention may be made, in a non-limiting manner, of ethynyl, prop-2-ynyl, but-2-ynyl, but-3-ynyl, pent-2-ynyl, pent-3-ynyl and pent-4-ynyl radicals;
the term “alkoxy” refers to the term “alkyl-oxy”;
the term “halogen” refers, in a non-limiting manner, to fluorine, chlorine or bromine;
the term “(C6-C14)aryl” refers to an aromatic group containing from 6 to 14 carbon atoms with at least one of the rings having a system of conjugated pi electrons, and including biaryls, which may be optionally substituted. Mention will be made in particular of biphenyl, phenyl, naphthyl, anthryl and phenanthryl radicals;
the term “hetero(C6-C14)aryl” refers to a 6-14-membered aromatic heterocycle containing 1-4 heteroatoms, the other atoms being carbon atoms. Among the heteroatoms, mention will be made in particular of oxygen, sulfur and nitrogen. Among the heteroaryl radicals, mention will be made more particularly of furyl, thienyl, pyridyl, pyrrolyl, pyrimidyl, pyrazinyl, oxazolyl, oxadiazolyl, isoxazolyl, quinolyl and thiazolyl radicals;
the term “(C3-C8)cycloalkyl” refers to a saturated hydrocarbon-based ring and contains monocyclic, bicyclic and polycyclic radicals containing from 3 to 8 carbon atoms. Mention will be made, in a non-limiting manner, of cyclopropyl and cyclobutyl radicals;
the term “(C6-C14)aryl(C1-C20)alkyl” refers to the corresponding -alkylaryl groups. Mention will be made in particular of benzyl and phenethyl groups.

It will be appreciated that the compounds that are useful according to the present invention may contain asymmetric centres. These asymmetric centres may be, independently, in R or S configuration. It will be clear to a person skilled in the art that certain compounds that are useful according to the invention may also exhibit geometrical isomerism. It should be understood that the present invention includes individual geometrical isomers and stereoisomers to and mixtures thereof, including racemic mixtures, of compounds of the formula (I) above. Isomers of this type can be separated from mixtures thereof by application or adaptation of known processes, for example chromatography techniques or recrystallisation techniques, or they are prepared separately from suitable isomers of their intermediates.

The enantiomers of the compounds according to the invention and the process for the preparation of them are especially described in patent application WO 2004/089917, the content of which is incorporated herein by reference.

The present patent application also concerns the polymorphic forms of the compounds, as obtained according to patent application WO 2004/089917, for instance the A1 polymorphic form of the salt (+)-2-amino-3,6-dihydro-4-dimethylamino-6-methyl-1,3,5-triazine hydrochloride.

The present invention also relates to the other polymorphic forms of the compounds, such as the H1 polymorphic form of the salt (+)-2-amino-3,6-dihydro-4-dimethylamino-6-methyl-1,3,5-triazine hydrochloride, which can be prepared as follows:

Approximately 3 g of the A1 form of Example 18 are dissolved in 50 ml of 1 mol/l HCl at room temperature. The clear solution obtained is left to evaporate at room temperature, in an open beaker, until a solid residue crystallises.

The characterisation is performed by:

FT-IR spectroscopy:
Brüker Vector 22
2 cm⁻¹ spectral resolution
32 scans
KBR discs (analogous to method A AA21505)
To evaluate the intensity of the IR bands, the IR spectra were normalised by vectorisation in the spectral range 4000-400 cm⁻¹ as an absorption spectrum.

Preadjustment was performed:
s: A>0.05
m: 0.01<A<0.05
w: A<0.01.

FT-Raman spectroscopy:
Brüker RFS-100
excitation: 1064 nm
spectral resolution: 1 cm⁻¹
1000 mW
1000 scans
focalised
aluminium crucible (analogous to method RA AA21505)
To evaluate the intensity of the Raman bands, Raman spectra were normalised by vectorisation in the spectral range 3600-200 cm⁻¹. Preadjustment was performed:
   s: A>0.05
   m: 0.01<A<0.05
   w: A<0.01

Powder x-ray diffraction (XRD)
diffractometer D5000 (Brüker AXS)
radiation CuKα1 at 1.5406 Å (U=30 kV, A=40 mA)
Transmission mode
Detector in sensitive position
Primary monochromator
Angle range: 3-65° 2θ
Stage width: 0.05° 2θ
Measuring time/stage: 1.4 s
The XRD machine is set at 2θ±0.1°.

### Results

A1 form:

### XRD:

| No. | d[Å] | 2θ | I/Io |
|---|---|---|---|
| 1 | 5.98 | 14.8 | 85 |
| 2 | 5.26 | 16.8 | 83 |
| 3 | 4.35 | 20.4 | 30 |
| 4 | 3.57 | 24.9 | 100 |
| 5 | 3.50 | 25.4 | 53 |
| 6 | 3.36 | 26.5 | 96 |
| 7 | 3.31 | 26.9 | 52 |
| 8 | 3.04 | 29.3 | 57 |
| 9 | 2.90 | 30.8 | 30 |
| 10 | 2.74 | 32.7 | 35 |

FT-IR bands (in cm⁻¹):
3384+/−1.5 (m), 3199+/−1.5 (m), 3163+/−1.5 (m), 3107+/−1.5 (m), 2993+/−1.5 (m), 2983+/−1.5 (m), 1652+/−1.5 (s), 1606+/−1.5 (s), 1576+/−1.5 (s), 1557+/−1.5 (s), 1505+/−1.5 (s), 1449+/−1.5 (m), 1427+/−1.5 (m), 1405+/−1.5 (m), 1383+/−1.5 (m), 1348+/−1.5 (m), 1306+/−1.5 (m), 1263+/−1.5 (w), 1235+/−1.5 (w), 1185+/−1.5 (w), 1096+/−1.5 (w), 1068+/−1.5 (w), 980+/−1.5 (w), 946+/−1.5 (w), 868+/−1.5 (w), 761+/−1.5 (w), 687+/−1.5 (m), 655+/−1.5 (m), 558+/−1.5 (w), 521+/−1.5 (w), 478+/−1.5 (w)
FT-Raman bands (in cm⁻¹):
3217+/−1.5 (w), 2994+/−1.5 (m), 2983+/−1.5 (m), 2936+/−1.5 (s), 2883+/−1.5 (m), 1645+/−1.5 (w), 1602+/−1.5 (m), 1554+/−1.5 (m), 1453+/−1.5 (m), 1428+/−1.5 (m), 1349+/−1.5 (w), 1308+/−1.5 (w), 979+/−1.5 (m), 866+/−1.5 (w), 761+/−1.5 (w), 686+/−1.5 (s), 583+/−1.5 (m), 555+/−1.5 (s), 525+/−1.5 (m), 479+/−1.5 (m), 410+/−1.5 (m), 401+/−1.5 (m), 307+/−1.5 (m)
H1 form

### XRD:

| No. | d[Å] | 2θ | I/Io |
|---|---|---|---|
| 1 | 8.03 | 11.0 | 69 |
| 2 | 7.27 | 12.2 | 25 |
| 3 | 6.11 | 14.5 | 24 |
| 4 | 4.01 | 22.1 | 86 |
| 5 | 3.64 | 24.5 | 100 |
| 6 | 3.26 | 27.3 | 51 |
| 7 | 3.08 | 29.0 | 29 |
| 8 | 3.04 | 29.4 | 34 |
| 9 | 2.82 | 31.7 | 61 |
| 10 | 2.66 | 33.6 | 26 |

FT-IR bands (in cm⁻¹):
3386+/−1.5 (m), 3080+/−3 (m), 1706+/−1.5 (s), 1691+/−1.5 (s), 1634+/−1.5 (m), 1513+/−1.5 (m), 1445+/−1.5 (w), 1241+/−1.5 (w), 1079+/−1.5 (w), 989+/−1.5 (w), 940+/−1.5 (w), 861+/−1.5 (w), 823+/−1.5 (w), 675+/−1.5 (w), 603+/−1.5 (w), 573+/−1.5 (w), 549+/−1.5 (w), 527+/−1.5 (w)

For the purposes of this text, it is understood that the tautomeric forms are included in the mention of a given group, for example thio/mercapto or oxo/hydroxy.

The pharmaceutical compositions according to the present invention are useful in the treatment of pathologies associated with insulin resistance syndrome (syndrome X).

Insulin resistance is characterised by a reduction in the action of insulin (cf. Presse Médicale, 1997, 26 (No. 14), 671-677) and is involved in a large number of pathological conditions, such as diabetes and more particularly non-insulin-dependent diabetes (type II diabetes or NIDDM), dyslipidaemia, obesity and arterial hypertension, and also certain microvascular and macrovascular complications, for instance atherosclerosis, retinopathy and neuropathy.

In this respect, reference will be made, for example, to Diabetes, vol. 37, 1988, 1595-1607; Journal of Diabetes and its Complications, 1998, 12, 110-119 or Horm. Res., 1992, 38, 28-32.

The aim of the present invention is to propose a pharmaceutical composition for significantly improving the condition of diabetics.

The pharmaceutical compositions of the invention especially have hypoglycaemiant and hypolipidaemiant activity.

The compounds of the formula (I) are therefore useful in the treatment of pathologies associated with hyperglycaemia and dyslipidaemia.

The pharmaceutical composition comprising the triazine compound of the formula (I) in combination with a PPARα agonist can be prepared by mixing together the various active principles, either all together or independently with a physiologically acceptable support, an excipient, a binder, a diluent, etc. It is then administered orally or non-orally, for instance via the parenteral, intravenous, cutaneous, nasal or rectal route. If the active principles are formulated independently, the corresponding formulations can be mixed together extemporaneously using a diluent and are then administered or can be administered independently of each other, either successively or sequentially.

The pharmaceutical compositions of the invention include formulations, such as granules, powders, tablets, gel capsules, syrups, emulsions and suspensions, and also forms used for non-oral administration, for instance injections, sprays or suppositories.

The pharmaceutical forms can be prepared via the known conventional techniques.

The preparation of an orally administered solid pharmaceutical form will be performed by the following process: an excipient (for example lactose, sucrose, starch, mannitol, etc.), a disintegrant (for example calcium carbonate, calcium carboxymethylcellulose, alginic acid, sodium carboxymethylcellulose, colloidal silicon dioxide, sodium croscarmellose, Crospovidone, guar gum, magnesium aluminium silicate, microcrystalline cellulose, cellulose powder, pregelatinised starch, sodium alginate, starch glycolate, etc.), a binder (for example alpha-starch, gum arabic, carboxymethylcellulose, polyvinylpyrrolidone, hydroxypropylcellulose, alginic acid, carbomer, dextrin, ethylcellulose, sodium alginate, maltodextrin, liquid glucose, magnesium aluminium silicate, hydroxyethylcellulose, methylcellulose, guar gum, etc.) and a lubricant (for example talc, magnesium stearate, polyethylene 6000, etc.) are, for example, added to the active principle(s) and the mixture obtained is then tabletted. If necessary, the tablet can be coated via the known techniques, in order to mask the taste (for example with cocoa powder, mint, borneol, cinnamon powder, etc.) or to allow enteric dissolution or sustained release of the active principles. The coating products that can be used are, for example, ethylcellulose, hydroxymethylcellulose, polyoxyethylene glycol, cellulose acetophthalate, hydroxypropylmethylcellulose phthalate and Eudragit® (methacrylic acid-acrylic acid copolymer), Opadry® (hydroxypropylmethylcellulose+macrogol+titanium oxide+lactose monohydrate). Pharmaceutically acceptable colorants may be added (for example yellow iron oxide, red iron oxide, quinoline yellow lake, etc.). Pharmaceutical forms, such as tablets, powders, sachets and gel capsules can be used for an oral administration.

The liquid pharmaceutical forms for oral administration include solutions, suspensions and emulsions. The aqueous solutions can be obtained by dissolving the active principles in water, followed by addition of flavourings, colorants, stabilisers and thickener, if necessary. In order to improve the solubility, it is possible to add ethanol, propylene glycol or other pharmaceutically acceptable non-aqueous solvents. The aqueous suspensions for oral use can be obtained by dispersing the finely divided active principles in water with a viscous product, such as natural or synthetic gums, resins, methylcellulose or sodium carboxymethylcellulose.

The pharmaceutical forms for injection can be obtained, for example, by the following process. The active principle(s) is (are) dissolved, suspended or emulsified either in an aqueous medium (for example distilled water, physiological saline, Ringer's solution, etc.) or in an oily medium (for example a plant oil, such as olive oil, sesameseed oil, cottonseed oil, corn oil, etc., or propylene glycol), with a dispersant (for example Tween 80, HCO 60 (Nikko Chemicals), polyethylene glycol, carboxymethylcellulose, sodium alginate, etc.), a preserving agent (for example methyl p-hydroxybenzoate, propyl p-hydroxybenzoate, benzyl alcohol, chlorobutanol, phenol, etc.), an isotonicity agent (for example sodium chloride, glycerol, sorbitol, glucose, etc.) and also other additives, such as, if desired, a solubilising agent (for example sodium salicylate, sodium acetate, etc.) or a stabiliser (for example human serum albumin).

A pharmaceutical form for external use can be obtained from a solid, semi-solid or liquid composition containing the active principle(s). For example, to obtain a solid form, the active principle(s) is (are) treated, alone or as mixtures, with excipients (for example lactose, mannitol, starch, microcrystalline cellulose, sucrose, etc.) and a thickener (for example natural gums, cellulose derivatives, acrylic polymers, etc.) so as to convert them into powder. The liquid pharmaceutical compositions are prepared in substantially the same way as the forms for injection, as indicated previously. The semi-solid pharmaceutical forms are preferably in the form of aqueous or oily gels or in the form of a pomade. These compositions may optionally contain a pH regulator (for example carbonic acid, phosphoric acid, citric acid, hydrochloric acid, sodium hydroxide, etc.) and a preserving agent (for example p-hydroxybenzoic acid esters, chlorobutanol, benzalkonium chloride, etc.) and also other additives.

The daily dose of PPARα agonist is between 50 mg and 2000 mg and that of the compounds of the formula (I) is between 200 mg and 2000 mg per day. This dose will depend on the patient and will be adapted in consequence.

The relative proportion of the constituents in the pharmaceutical compositions of the present invention takes into account the recommended dosages of the respective active principles. These relative proportions of the PPARα agonists, or of pharmaceutically acceptable salts thereof, and of the compounds of the formula (I), or of pharmaceutically acceptable salts thereof, thus vary in consequence. For example, the weight ratio of the compound of the formula (I) relative to the PPARα agonist may range between 1/1 and 20/1 and preferably from 2/1 to 5/1. The frequency of administration of the compounds of the invention is between one and two administrations per day. In the case where the doses of compounds of the formula (I) necessitate more than one daily administration, the amounts of PPARα agonist and the PPARα agonist/compound of the formula (I) ratio will be adjusted in consequence.

The aim of the present invention is also to propose a method of treatment via co-administration of an effective amount of a compound of the formula (I) and of a PPARα agonist, and also kits for allowing this co-administration.

The present invention also relates to kits that are suitable for the treatment by the methods described above. These kits comprise a composition containing the compound of the formula (I) in the dosages indicated above and a second composition containing the PPARα agonist in the dosages indicated above, for a simultaneous, separate or sequential administration, in effective amounts according to the invention.

The term “co-administration” means the simultaneous, separate or sequential administration of one or more compounds to the same patient, over a period that may be up to 2 hours or even up to 12 hours. For example, the term co-administration includes (1) a simultaneous administration of the two compounds, (2) an administration of the first, followed 2 hours later by the administration of the second compound, (3) an administration of the first, followed 12 hours later by the administration of the second compound.

The examples below of compositions according to the invention are given as non-limiting illustrations.

### EXAMPLES

The amounts are expressed on a weight basis.

### Formulation Example 1

(+)-2-amino-3,6-dihydro-4-dimethylamino-6-methyl-1,3,5-triazine hydrochloride: 1000 mg
fenofibrate: 100 mg
microcrystalline cellulose: 110 mg
croscarmellose: 28 mg
polyvinylpyrrolidone: 40 mg
magnesium stearate: 14 mg
Opadry: 24 mg

### Formulation Example 2

(+)-2-amino-3,6-dihydro-4-dimethylamino-6-methyl-1,3,5-triazine hydrochloride: 1000 mg
fenofibrate: 50 mg
microcrystalline cellulose: 60 mg
croscarmellose: 28 mg
polyvinylpyrrolidone: 40 mg
magnesium stearate: 9 mg
Opadry®: 24 mg

### Formulation Example 3

(+)-2-amino-3,6-dihydro-4-dimethylamino-6-methyl-1,3,5-triazine hydrochloride: 750 mg
gemfibrozil: 200 mg
microcrystalline cellulose: 60 mg
croscarmellose: 21 mg
polyvinylpyrrolidone: 30 mg
magnesium stearate: 10.5 mg
Opadry®: 18 mg

Biological Results for the Combinations According to the Invention

Male homozygous mice C57BUKs/Ola/Hsd/lep ob/ob are reared for two weeks in a room at controlled temperature, humidity and light (21-23° C., 12-12 hour day-night cycles). They are fed on a standard laboratory diet and given free access to water. After acclimatisation, they are randomised into groups of 10 on the basis of body weight, as follows:
vehicle: untreated mice,
group A: mice treated once a day with the hydrochloride salt of (+)-2-amino-3,6-dihydro-4-dimethylamino-6-methyl-1,3,5-triazine at a rate of 100 mg/kg,
PPARα 100 group: mice treated once a day with a PPARα agonist at a rate of 100 mg/kg,
PPARα 100+ compound 100 group: mice treated once a day with a PPARα agonist at a rate of 100 mg/kg and the hydrochloride salt of (+)-2-amino-3,6-dihydro-4-dimethylamino-6-methyl-1,3,5-triazine at a rate of 100 mg/kg.

The serum triglyceride levels (expressed in g/L) are measured at the start and end of the study for each group.

1. Pharmaceutical composition comprising, as active principle:
i) a PPARα agonist,
ii) a triazine derivative of the formula (I) 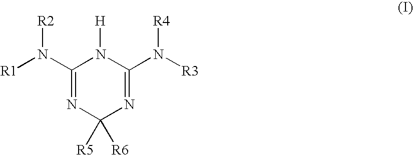
in which:
R1, R2, R3 and R4 are independently chosen from the following groups:
H,
(C1-C20)alkyl optionally substituted by halogen, (C1-C5)alkyl, (C1-C5)alkoxy or (C3-C8)cycloalkyl,
(C2-C20)alkenyl optionally substituted by halogen, (C1-C5)alkyl or (C1-C5)alkoxy
(C2-C20)alkynyl optionally substituted by halogen, (C1-C5)alkyl or (C1-C5)alkoxy
(C3-C8)cycloalkyl optionally substituted by (C1-C5)alkyl or (C1-C5)alkoxy
hetero(C3-C8)cycloalkyl bearing one or more heteroatoms chosen from N, O and S and optionally substituted by (C1-C5)alkyl or (C1-C5)alkoxy
(C6-C14)aryl(C1-C20)alkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C6-C14)aryl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C1-C13)heteroaryl bearing one or more heteroatoms chosen from N, O and S and optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
R1 and R2, on the one hand, and R3 and R4, on the other hand, possibly forming with the nitrogen atom an n-membered ring (n between 3 and 8) optionally containing one or more heteroatoms chosen from N, O and S and possibly being substituted by one or more of the following groups: amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
R5 and R6 are independently chosen from the following groups:
H,
(C1-C20)alkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C2-C20)alkenyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C2-C20)alkynyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C3-C8)cycloalkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
hetero(C3-C8)cycloalkyl bearing one or more heteroatoms chosen from N, O and S and optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C6-C14)aryl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C1-C13)heteroaryl bearing one or more heteroatoms chosen from N, O and S and optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C6-C14)aryl(C1-C5)alkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
R5 and R6 possibly forming with the carbon atom to which they are attached an m-membered ring (m between 3 and 8) optionally containing one or more heteroatoms chosen from N, O and S and possibly being substituted by amino, hydroxyl, thio, halogen, (C1-C5)alkyl, (C1-C5)alkoxy, (C1-C5)-alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
or possibly forming with the carbon atom a C10-C30 polycyclic residue optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
R5 and R6 together also possibly representing the group ═O or ═S, the nitrogen atom of a heterocycloalkyl or heteroaryl group possibly being substituted by a (C1-C5)alkyl, (C3-C8)cycloalkyl, (C6-C14)aryl, (C6-C14)aryl(C1-C5)alkyl or (C1-C6)acyl group,
and also the racemic forms, tautomers, enantiomers, diastereoisomers, epimers and polymorphs, and mixtures thereof, and the pharmaceutically acceptable salts,
in combination with one or more pharmaceutically acceptable excipients.

2. Pharmaceutical composition according to claim 1, comprising a compound of the formula (I) in which R5 is hydrogen.

3. Pharmaceutical composition according to claim 1, comprising a compound of the formula (I) in which R5 and R6 are independently chosen from H and (C1-C20)alkyl groups optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl.

4. Pharmaceutical composition according to claim 1, comprising a compound of the formula (I) in which R1, R2, R3 and R4 are independently chosen from H and (C1-C20)alkyl groups optionally substituted by halogen, (C1-C5)alkyl, (C1-C5)alkoxy or (C3-C8)cycloalkyl.

5. Pharmaceutical composition according to claim 1, comprising a compound of the formula (I) in which R5 and R6 are independently chosen from H and (C1-C20)alkyl groups optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxy, carboxymethyl or carboxyethyl; more preferably, R5=H and R6=(C1-C20)alkyl optionally substituted by amino, hydroxyl, thio, halogen, (C1-C5)alkyl, (C1-C5)alkoxy, (C1-C5)alkylthio, (C1-C5)alkylamino, (C6-C14)-aryloxy, (C6-C14)aryl(C1-C5)alkoxy, cyano, trifluoromethyl, carboxyl, carboxymethyl or carboxyethyl, or vice versa.

6. Pharmaceutical composition according to claim 1, comprising a compound of the formula (I) in which R1 and R2 are a methyl group and R3 and R4 represent a hydrogen.

7. Pharmaceutical composition according to Claim 1, characterised in that the compound of the formula (I) is 2-amino-3,6-dihydro-4-dimethylamino-6-methyl-1,3,5-triazine, and also the racemic forms, tautomers, enantiomers, diastereoisomers, epimers and mixtures thereof, and the pharmaceutically acceptable salts.

8. Pharmaceutical composition according to claim 1, characterised in that the compound of the formula (I) is (−)-2-amino-3,6-dihydro-4-dimethylamino-6-methyl-1,3,5-triazine, and also the racemic forms, tautomers, enantiomers, diastereoisomers, epimers and mixtures thereof, and the pharmaceutically acceptable salts.

9. Pharmaceutical composition according to claim 1, characterised in that the compound of the formula (I) is (+)-2-amino-3,6-dihydro-4-dimethylamino-6-methyl-1,3,5-triazine, and also the racemic forms, tautomers, enantiomers, diastereoisomers, epimers and mixtures thereof, and the pharmaceutically acceptable salts.

10. Pharmaceutical composition according to claim 1, such that the compound of the formula (I) is in the form of a hydrochloride.

11. Pharmaceutical composition according to claim 1, characterised in that the PPARα agonists are in the form of a pharmaceutically acceptable salt.

12. Pharmaceutical composition according to claim 1, characterised in that these pharmaceutical compositions contain between 50 mg and 600 mg of PPARα agonist.

13. Pharmaceutical composition according to claim 1, characterised in that these pharmaceutical compositions contain between 200 mg and 2000 mg of compound of the formula (I).

14. Pharmaceutical composition according to claim 1, characterised in that the weight ratio of the PPARα agonists to the compound of the formula (I) is between 1/1 and 1/20.

15. Pharmaceutical composition according to claim 1, characterised in that the PPARα agonist is chosen from fenofibrate, bezafibrate, gemfibrozil and ciprofibrate.

16. Pharmaceutical composition according to claim 1, characterised in that the PPARα agonist is fenofibrate and the compound of the formula (I) is (+)-2-amino-3,6-dihydro-4-dimethylamino-6-methyl-1,3,5-triazine, optionally in the form of a hydrochloride.

17. Pharmaceutical composition according to claim 1, characterised in that the PPARα agonist is bezafibrate and the compound of the formula (I) is (+)-2-amino-3,6-dihydro-4-dimethylamino-6-methyl-1,3,5-triazine, optionally in the form of a hydrochloride.

18. Pharmaceutical composition according to claim 1, characterised in that the PPARα agonist is gemfibrozil and the compound of the formula (I) is (+)-2-amino-3,6-dihydro-4-dimethylamino-6-methyl-1,3,5-triazine, advantageously in the form of a hydrochloride.

19. Pharmaceutical composition according to claim 1, characterised in that the PPARα agonist is ciprofibrate and the compound of the formula (I) is (+)-2-amino-3,6-dihydro-4-dimethylamino-6-methyl-1,3,5-triazine, optionally in the form of a hydrochloride.

20. Pharmaceutical composition according to claim 1, which is suitable for oral administration, in which the pharmaceutical composition is a powder, a coated tablet, a gel capsule, a sachet, a solution, a suspension or an emulsion.

21. A method for the treatment of and/or preventing diabetes comprising administering to a subject in need thereof an effective amount of a PPARα agonist in combination with an effective amount of a compound of the formula (I) according to claim 1.

22. A method according to claim 21, for the treatment of and/or preventing non-insulin-dependent diabetes.

23. A method for the treatment of a pathology associated with insulin resistance syndrome, chosen from dyslipidaemia, obesity, arterial hypertension, and microvascular and macrovascular complications atherosclerosis, retinopathy, nephropathy and neuropathy comprising administering to a subject in need thereof an effective amount of a PPARα agonist in combination with an effective amount of a compound of the formula (I) according to claim 1.

24. A method according to claim 21, wherein the PPARα agonist is fenofibrate, bezafibrate, gemfibrozil or ciprofibrate.

25. A method according to claim 24, wherein the compound of the formula (I) is (+)-2-amino-3,6-dihydro-4-dimethylamino-6-methyl-1,3,5-triazine, optionally in the form of a hydrochloride.

26. A method according to claim 21, wherein the administration of the compound of the formula (I) and that of the PPARα agonist are simultaneous, separate or sequential.

27. Kit comprising a compound of the formula (I) according to claim 1 and a PPARα agonist which is fenofibrate, bezafibrate, gemfibrozil or ciprofibrate, for simultaneous, separate or sequential administration.
